SEQUENCE LISTING (25 sequences)

SEQ ID NO: 1 - protein, 10 aa, Homo sapiens
GPPGPPGRLV

SEQ ID NO: 2 - protein, 6 aa, Homo sapiens
PPGRLV

SEQ ID NO: 3 - protein, 11 aa, synthetic construct
  REGION 1..11: note = C-terminal neo-epitope elongated
  VAR_SEQ 11: note = X is one or more amino acids of the sequence of collagen type VII alpha 1
GPPGPPGRLVX

SEQ ID NO: 4 - protein, 10 aa, Homo sapiens
EAPRVRAQHR

SEQ ID NO: 5 - protein, 6 aa, Homo sapiens
EAPRVR

SEQ ID NO: 6 - protein, 11 aa, synthetic construct
  VAR_SEQ 1: note = X is one or more amino acids of thr sequence of collagen type VII alpha 1
  REGION 1..11: note = N-terminal neo-epitope elongated
XEAPRVRAQHR

SEQ ID NO: 7 - protein, 10 aa, synthetic construct
  SITE 1: note = Biotinylated with optional linker between biotin residue and peptide
  REGION 1..10: note = C-terminal neo-epitope - biotinylated at N-terminus
GPPGPPGRLV

SEQ ID NO: 8 - protein, 10 aa, synthetic construct
  REGION 1..10: note = N-terminal neo-epitope - biotinylated at C-terminus
  SITE 10: note = Biotinylated with optional linker between biotin residue and peptide
EAPRVRAQHR

SEQ ID NO: 9 - protein, 13 aa, synthetic construct
  SITE 1: note = KLH bound to CGG linker
  REGION 1..13: note = immunisation peptide
CGGGPPGPPGRLV

SEQ ID NO: 10 - protein, 12 aa, synthetic construct
  SITE 1: note = Biotinylated
  REGION 1..12: note = Coater peptide
KKGPPGPPGRLV

SEQ ID NO: 11 - protein, 11 aa, synthetic construct
  REGION 1..11: note = elongated C-terminal sequence
GPPGPPGRLVD

SEQ ID NO: 12 - protein, 13 aa, synthetic construct
  REGION 1..13: note = Immunisation peptide
  SITE 13: note = KLH bound
EAPRVRAQHRGGC

SEQ ID NO: 13 - protein, 11 aa, synthetic construct
  REGION 1..11: note = Coater peptide
  SITE 11: note = Biotinylated
EAPRVRAQHRK

SEQ ID NO: 14 - protein, 16 aa, Mus musculus
RSSQSLLYSNGITYLY

SEQ ID NO: 15 - protein, 7 aa, Mus musculus
QMSNLAS

SEQ ID NO: 16 - protein, 9 aa, Mus musculus
AQNLELPWT

SEQ ID NO: 17 - protein, 5 aa, Mus musculus
ENTMN

SEQ ID NO: 18 - protein, 17 aa, Mus musculus
GINPKIGGSIYNQKFQD

SEQ ID NO: 19 - protein, 11 aa, Mus musculus
GIYDEGWHFDV

SEQ ID NO: 20 - protein, 79 aa, Mus musculus
RSSQSLLYSNGITYLYWYLQKPGQSPQLLIYQMSNLASGVPDRFSSSGSGTDFTLRISRVEAEDVGVYYCAQNLELPWT

SEQ ID NO: 21 - protein, 79 aa, Mus musculus
ENTMNWVKQSHGKSLEWIGGINPKIGGSIYNQKFQDKATLTVDKSSSTAYMELRSLTSDDSAVYYCARGIYDEGWHFDV

SEQ ID NO: 22 - protein, 112 aa, Mus musculus
DIVMTQAAFSNPVTLGTSASISCRSSQSLLYSNGITYLYWYLQKPGQSPQLLIYQMSNLASGVPDRFSSSGSGTDFTLRISRVEAEDVGVYYCAQNLELPWTFGGGTKLEIK

SEQ ID NO: 23 - protein, 120 aa, Mus musculus
EVQLQQSGPELVKPGASVKISCKTSGYTFIENTMNWVKQSHGKSLEWIGGINPKIGGSIYNQKFQDKATLTVDKSSSTAYMELRSLTSDDSAVYYCARGIYDEGWHFDVWGAGTTVTVSS

SEQ ID NO: 24 - protein, 463 aa, Mus musculus
MGWSWIFLFLLSGTAGVLSEVQLQQSGPELVKPGASVKISCKTSGYTFIENTMNWVKQSHGKSLEWIGGINPKIGGSIYNQKFQDKATLTVDKSSSTAYMELRSLTSDDSAVYYCARGIYDEGWHFDVWGAGTTVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 25 - protein, 239 aa, Mus musculus
MRFSAQLLGLLVLWIPGSTADIVMTQAAFSNPVTLGTSASISCRSSQSLLYSNGITYLYWYLQKPGQSPQLLIYQMSNLASGVPDRFSSSGSGTDFTLRISRVEAEDVGVYYCAQNLELPWTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

(19) United States
(12) Patent Application Publication
(10) Pub. No.: US 2025/0012810 A1
(43) Pub. Date: Jan. 9, 2025

(54) COLLAGEN TYPE VII ALPHA 1 ASSAY

(71) Applicant: NORDIC BIOSCIENCE A/S, Herlev (DK)

(72) Inventors: Jannie Marie Bülow SAND, Måløv (DK); Diana Julie LEEMING, Espergærde (DK); Morten KARSDAL, København Ø (DK)

(73) Assignee: NORDIC BIOSCIENCE A/S, Herlev (DK)

(21) Appl. No.: 18/765,104

(22) Filed: Jul. 5, 2024

Related U.S. Application Data
(63) Continuation-in-part of application No. 17/339,317, filed on Jun. 4, 2021, now abandoned.
Application No. 17/339,317 is a continuation of application No. 16/072,648, filed on Jul. 25, 2018, now abandoned.

(30) Foreign Application Priority Data
Jan. 28, 2016 (GB) 1601571.1

(51) Int. Cl. G01N 33/68; C07K 14/78; C07K 16/18; G01N 33/564

(52) U.S. Cl. CPC G01N 33/6887; C07K 14/78; C07K 16/18; G01N 33/564; G01N 2333/78; G01N 2800/12; G01N 2800/122

(57) ABSTRACT

A method of immunoassay for detecting in a biological sample a fragment of collagen type VII alpha 1 comprising an N- or C-terminal neo-epitope, the method comprising contacting the biological sample comprising the fragment of collagen type VII alpha 1 comprising the N- or C-terminal neo-epitope with an antibody of the invention, and determining the amount of binding of the antibody

### SEQUENCE ID INCORPORATION BY REFERENCE

The entire content of the following electronic submission of the sequence listing via the USPTO EFS-WEB server, as authorized and set forth in 37 CFR 1.835(a) (2) is incorporated herein by reference in its entirety for all purposes. The submission of this replacement sequence listing removes the previously submitted “SequenceListing_350010-1011CCIP” PDF. The sequence listing is within the electronically filed xml filed that is identified as follows:
File Name: “P19874US-2 Sequence Listing. xml”
Date of Creation: Sep. 23, 2024
Size: 30,000 bytes.

### TECHNICAL FIELD

The present invention relates to antibodies which are reactive with fragments of collagen type VII alpha 1 comprising an N- or C-terminal neo-epitope, the use of said antibodies in an assay for detecting and quantifying said fragments of collagen type VII alpha 1, and the use of said assay for evaluating chronic obstructive pulmonary disease (COPD) or systemic sclerosis.

### BACKGROUND ART

Collagen type VII is the main component of the anchoring fibrils that connects the basement membrane to the underlying interstitial matrix. It consists of three identical alpha-1 chains with two non-collagenous (NC) domains and a central collagenous triple helical domain. It has been identified in the basement membranes of skin and mucous membranes [1].

Collagen type VII has mainly been investigated for its role in dystrophic epidermolysis bullosa, a severe skin disease. Mutations in the collagen type VII alpha-1 chain leads to the formation of abnormal, diminished or absent anchoring fibrils which causes separation of epidermis from dermis and thus skin blistering [1]. Collagen type VII has also been identified as the protein at fault in epidermolysis bullosa acquisita, an autoimmune disease causing blistering of the skin and mucous membranes. It is caused by IgG autoantibodies directed at the collagen type VII NC1 domain [2].

Autoimmunity to collagen type VII has also been associated with inflammatory bowel disease and bullous systemic lupus erythematosus [3-4].

An up-regulation of collagen type VII level in the skin of patients suffering from systemic sclerosis has been identified [5]. Patients with systemic sclerosis have skin fibrosis and may present with fibrosis of internal organs including the lungs. One study has also identified a reduced level of collagen type VII protein in the anchoring fibrils in the airways in a monkey model of asthma [6].

In order to evaluate a pathogenic condition linked to collagen type VII it is necessary to produce assays capable of detecting and quantifying species related to the pathogenic condition.

Chen et al., Saleh et al. and Kim et al. independently developed ELISAs for detecting autoantibodies against the NC1 or NC2 domain of collagen type VII [7-9]. The methods comprise coating a microtiter plate with recombinant NC1 and/or NC2 domain of collagen type VII alpha-1, adding serum samples of interest, and using anti-human IgG antibody to detect autoantibodies present in the serum sample.

Recke et al. describes the generation of autoantibodies against the collagen type VII NC1 domain and investigated the pathogenic potential in human ex vivo models of epidermolysis bullosa acquisita [10]. They proposed the use of this as a diagnostic tool.

Sakai et al. raised a monoclonal antibody against collagen type VII [11]. The mAb was reactive only with intact collagen type VII.

Monoclonal and polyclonal antibodies targeting collagen type VII can be obtained commercially from several vendors.

It has now been found that fragments of collagen type VII alpha 1 are detectable in circulation and could serve as potential biomarkers for evaluating pathological conditions linked to collagen type VII. Specifically, a link between collagen type VII alpha 1 fragments and the pathological conditions COPD and systemic sclerosis has been identified.

### SUMMARY OF THE INVENTION

Accordingly, in a first aspect the present invention relates to an antibody reactive with a fragment of collagen type VII alpha 1 comprising an N- or C-terminal neo-epitope, wherein said antibody binds to the N- or C-terminal neo-epitope.

The antibody is preferably a monoclonal antibody, but may also be a polyclonal antibody or an antibody fragment exhibiting the desired biological activity.

Preferably, the antibody does not recognise or bind intact collagen type VII alpha 1.

In a preferred embodiment, the antibody may bind to an N- or C-terminal neo-epitope comprised in a non-collagenous amino-terminal domain of collagen type VII alpha 1 (NC1) or comprised in a central collagenous domain of collagen type VII alpha 1.

In another preferred embodiment, the antibody may bind to a C-terminal neo-epitope comprised in the central collagenous domain of collagen type VII alpha 1. Preferably, the antibody binds to a C-terminal neo-epitope comprised in the amino acid sequence GPPGPPGRLV-COOH (SEQ ID NO: 1).

Preferably, the antibody binds to a C-terminal neo-epitope comprising or consisting of the amino acid sequence PPGRLV-COOH (SEQ ID NO: 2). This C-terminal neo-epitope may be formed by cleavage of human collagen type VII alpha 1 at the Val-Asp bond between amino acids V1709-D1710 in the central collagenous domain of collagen type VII alpha 1. Preferably, the antibody does not recognise or bind elongated amino acid sequence GPPGPPGRLVX-COOH (SEQ ID NO: 3), wherein X is one or more amino acids of the sequence of collagen type VII alpha 1.

In another preferred embodiment, the antibody may bind to an N-terminal neo-epitope comprised in the non-collagenous amino-terminal domain of collagen type VII alpha 1 (NC1). Preferably, the antibody may bind to an N-terminal neo-epitope comprised in the amino acid sequence H₂N-EAPRVRAQHR (SEQ ID NO: 4). Preferably, the antibody binds to an N-terminal neo-epitope comprising or consisting of the amino acid sequence H₂N-EAPRVR (SEQ ID NO: 5). This N-terminal neo-epitope may be formed by cleavage of human collagen type VII alpha 1 at the Ala-Glu bond between amino acids A16-E17 in the non-collagenous amino-terminal domain of collagen type VII alpha 1. Preferably, the antibody does not recognise or bind elongated amino acid sequence H2N-XEAPRVRAQHR (SEQ ID NO: 6), wherein X is one or more amino acids of the sequence of collagen type VII alpha 1.

Antibodies described herein may be raised against a synthetic peptide corresponding to the N- or C-terminal neo-epitope amino acid sequence. Preferably the antibody is a monoclonal antibody.

As noted above, in certain exemplary embodiments the antibody is preferably a monoclonal antibody that binds to a C-terminal neo-epitope comprised in the amino acid sequence GPPGPPGRLV-COOH (SEQ ID NO: 1) and/or comprising or consisting of the amino acid sequence PPGRLV-COOH (SEQ ID NO: 2), and which antibody does not recognise or bind to the elongated amino acid sequence GPPGPPGRLVX-COOH (SEQ ID NO: 3) wherein X is one or more amino acids of the sequence of collagen type VII alpha 1.

In such exemplary embodiments, the monoclonal antibody may preferably comprise one or more complementarity-determining regions (CDRs) selected from:

CDR-L1:
(SEQ ID NO: 14)
RSSQSLLYSNGITYLY
CDR-L2:
(SEQ ID NO: 15)
QMSNLAS
CDR-L3:
(SEQ ID NO: 16)
AQNLELPWT
CDR-H1:
(SEQ ID NO: 17)
ENTMN
CDR-H2:
(SEQ ID NO: 18)
GINPKIGGSIYNQKFQD
CDR-H3:
(SEQ ID NO: 19)
GIYDEGWHFDV

Preferably the monoclonal antibody comprises at least 2, 3, 4, 5 or 6 of the above listed CDR sequences.

Preferably the monoclonal antibody has a light chain variable region comprising the CDR sequences:

CDR-L1:
(SEQ ID NO: 14)
RSSQSLLYSNGITYLY
CDR-L2:
(SEQ ID NO: 15)
QMSNLAS
and
CDR-L3:
(SEQ ID NO: 16)
AQNLELPWT.

Preferably the monoclonal antibody has a light chain that comprises framework sequences between the CDRs, wherein said framework sequences are substantially identical or substantially similar to the framework sequences between the CDRs in the light chain sequence below (in which the CDRs are shown in bold and underlined, and the framework sequences are shown in italics)

(SEQ ID NO: 20)
RSSQSLLYSNGITYLYWYLQKPGQSPQLLIYQMSNLASGVPDRFSSSGSG
TDFTLRISRVEAEDVGVYYCAQNLELPWT

Preferably the monoclonal antibody has a heavy chain variable region comprising the CDR sequences:

CDR-H1:
(SEQ ID NO: 17)
ENTMN
CDR-H2:
(SEQ ID NO: 18)
GINPKIGGSIYNQKFQD
and
CDR-H3:
(SEQ ID NO: 19)
GIYDEGWHFDV.

Preferably the monoclonal antibody has a heavy chain that comprises framework sequences between the CDRs, wherein said framework sequences are substantially identical or substantially similar to the framework sequences between the CDRs in the heavy chain sequence below (in which the CDRs are shown in bold and underlined, and the framework sequences are shown in italics)

(SEQ ID NO: 21)
ENTMNWVKQSHGKSLEWIGGINPKIGGSIYNQKFQDKATLTVDKSSSTAY
MELRSLTSDDSAVYYCARGIYDEGWHFDV

Preferably, the monoclonal antibody comprises the light chain variable region sequence:

(SEQ ID NO: 22)
DIVMTQAAFSNPVTLGTSASISCRSSQSLLYSNGITYLYWYLQKPGQSPQ
LLIYQMSNLASGVPDRFSSSGSGTDFTLRISRVEAEDVGVYYCAQNLELP
WTFGGGTKLEIK
(CDRs bold and underlined; Framework sequences in
italics) and/or the heavy chain variable region
sequence:
(SEQ ID NO: 23)
EVQLQQSGPELVKPGASVKISCKTSGYTFIENTMNWVKQSHGKSLEWIGG
INPKIGGSIYNQKFQDKATLTVDKSSSTAYMELRSLTSDDSAVYYCARGI
YDEGWHFDVWGAGTTVTVSS
(CDRs bold and underlined; Framework sequences in
italics)

As used herein, the framework amino acid sequences between the CDRs of an antibody are “substantially identical” or “substantially similar” to the framework amino acid sequences between the CDRs of another antibody if they have at least 70%, 80%, 90% or at least 95% similarity or identity. The similar or identical amino acids may be contiguous or non-contiguous. The framework sequences may contain one or more amino acid substitutions, insertions and/or deletions. Amino acid substitutions may be conservative, by which it is meant the substituted amino acid has similar chemical properties to the original amino acid. A skilled person would understand which amino acids share similar chemical properties. For example, the following groups of amino acids share similar chemical properties such as size, charge and polarity: Group 1 Ala, Ser, Thr, Pro, Gly; Group 2 Asp, Asn, Glu, Gln; Group 3 His, Arg, Lys; Group 4 Met, Leu, Ile, Val, Cys; Group 5 Phe Thy Trp.

A program such as the CLUSTAL program to can be used to compare amino acid sequences. This program compares amino acid sequences and finds the optimal alignment by inserting spaces in either sequence as appropriate. It is possible to calculate amino acid identity or similarity (identity plus conservation of amino acid type) for an optimal alignment. A program like BLASTx will align the longest stretch of similar sequences and assign a value to the fit. It is thus possible to obtain a comparison where several regions of similarity are found, each having a different score. Both types of analysis are contemplated in the present invention. Identity or similarity is preferably calculated over the entire length of the framework sequences.

In a second aspect the present invention relates to a method of immunoassay for detecting in a biological sample a fragment of collagen type VII alpha 1 comprising an N- or C-terminal neo-epitope, said method comprising contacting said biological sample comprising said fragment of collagen type VII alpha 1 comprising said N- or C-terminal neo-epitope with an antibody as described herein, and determining the amount of binding of said antibody.

The method may be used to quantify the amount of the fragment of collagen type VII alpha 1 comprising said N- or C-terminal neo-epitope in biofluids. The biofluid may be, but is not limited to, serum, plasma, bronchoalveolar lavage fluid, sputum, saliva, exhaled breath or urine.

The immunoassay may be, but is not limited to, a competition assay or a sandwich assay. The immunoassay may be, but is not limited to, a radioimmunoassay or an enzyme-linked immunosorbent assay.

The method may further comprise the step of correlating the quantity of the fragment of collagen type VII alpha 1 comprising an N- or C-terminal neo-epitope determined by said method with standard collagen type VII related disease samples of known disease severity to evaluate the severity of a collagen type VII related disease.

Such collagen type VII related diseases may be, but are not limited to, chronic obstructive pulmonary disease (COPD) or systemic sclerosis.

It is envisaged that the method of the invention may be utilised in the quantitation, diagnosis and/or prognosis of such collagen type VII related diseases.

In a third aspect the present invention relates to a peptide, wherein the peptide has an N-terminal amino acid sequence corresponding to an amino acid sequence of an N-terminal neo-epitope of a fragment of collagen type VII alpha 1 comprising said N-terminal neo-epitope, or wherein the peptide has a C-terminal amino acid sequence corresponding to an amino acid sequence of an C-terminal neo-epitope of a fragment of collagen type VII alpha 1 comprising said C-terminal neo-epitope. Preferably, the peptide is ten amino acid residues in length, more preferably nine amino acid residues, more preferably eight amino acid residues, more preferably seven amino acid residues, and most preferably six amino acid residues in length. The peptide may be biotinylated.

In a preferred embodiment, the peptide has the amino acid sequence EAPRVRAQHR (SEQ ID NO: 4) or EAPRVR (SEQ ID NO: 5).

In another preferred embodiment, the peptide has the amino acid sequence GPPGPPGRLV (SEQ ID NO: 1) or PPGRLV (SEQ ID NO: 2).

In a fourth aspect the present invention relates to an assay kit for determining the quantity of a fragment of collagen type VII alpha 1 comprising an N- or C-terminal neo-epitope in a biological sample, the kit comprising an antibody as described herein and at least one of:
a streptavidin coated 96 well plate
a biotinylated peptide corresponding to the amino acid sequence of the N- or C-terminal neo-epitope, with an optional linker located between the biotin residue and the peptide
a biotinylated secondary antibody for use in a sandwich immunoassay
a calibrator peptide corresponding to the amino acid sequence of the N- or C-terminal neo-epitope
an antibody HRP labeling kit
an antibody radiolabeling kit
an assay visualization kit

Preferably, the assay kit comprises a biotinylated peptide Biotin-L-GPPGPPGRLV (SEQ ID NO: 7), wherein L is an optional linker, and a calibrator peptide comprising the C-terminal sequence GPPGPPGRLV-COOH (SEQ ID NO: 1).

Preferably, the assay kit comprises a biotinylated peptide EAPRVRAQHR-L-Biotin (SEQ ID NO: 8), wherein L is an optional linker, and a calibrator peptide comprising the N-terminal sequence H₂N-EAPRVRAQHR (SEQ ID NO: 4).

### DEFINITIONS

The term “antibody” is used according to the invention in the broadest sense and specifically covers intact monoclonal antibodies, polyclonal antibodies, and antibody fragments, so long as they exhibit the desired biological activity (i.e. binding specificity for the target N- or C-terminal neo-epitope).

The term “monoclonal antibody” is used according to the invention in the broadest sense and specifically covers both intact monoclonal antibodies and antibody fragments, so long as they exhibit the desired biological activity (i.e. binding specificity for the target N- or C-terminal neo-epitope).

“Antibody fragments” according to the invention comprise a portion of an intact antibody, comprising the antigen-binding or variable region thereof. Examples of antibody fragments include Fab, Fab′, F(ab′)2, and Fv fragments.

“A fragment of Collagen type VII alpha 1” according to the invention means a peptide fragment produced by protease cleavage of collagen type VII.

“N- or C-terminal neo-epitope” according to the invention means an N- or C-terminal epitope formed at a protease cleavage site of Collagen type VII alpha 1. For example, the following sequence of Collagen type VII alpha 1

. . . PGPPGPPGRLV↓DTGPGAREKGE . . .

would produce the N-terminal neo-epitope H₂N-DTGPGAREKGE . . . and the C-terminal neo-epitope . . . PGPPGPPGRLV-COOH when cleaved by a protease at the site between the V¹⁷⁰⁹-D¹⁷¹⁰ peptide bond, as denoted by the symbol “↓”.

“C7” as used herein refers to fragments of collagen type VII alpha 1 comprising the C-terminal neo-epitope GPPGPPGRLV-COOH (SEQ ID NO: 1).

“NB677” as used herein refers to fragments of collagen type VII alpha 1 comprising the N-terminal neo-epitope H2N-EAPRVRAQHR (SEQ ID NO: 4).

### FIGURES

FIG. 1 shows a calibration curve for the “C7” assay.

FIG. 2 shows the correlation between “C7” and COPD.

FIG. 3 shows the correlation between “C7” and systemic sclerosis.

FIG. 4 shows a calibration curve for the “NB677” assay.

FIG. 5. Clinical evaluation of serum C7 in systemic sclerosis. Serum C7 levels were assessed in healthy donors (n=70) and a cohort of patients with systemic sclerosis (SSC; n=119). Data are presented as Tukey's box plots. Statistical significance was evaluated by Mann-Whitney test. *** p<0.0001.

### EXAMPLES

### Example 1—COPD Biomarker (“C7” Assay)

### Rationale

Mass spectrometry was performed on serum samples from a patient with COPD, a patient with idiopathic pulmonary fibrosis (IPF), and a healthy donor.

The initial mass spectrometry analyses identified peptides derived from collagen type VII in serum. Peptides were isolated from serum using IMAC Cu beads. Identity significance threshold for individual peptides were 51. Serum samples were analyzed using an orbitrap (OrbiB) instrument.

Fragments of collagen type VII alpha 1 comprising the C-terminal neo-epitope GPPGPPGRLV-COOH (“C7”) (SEQ ID NO: 1) were found in the COPD sample but not in the IPF or healthy donor samples. The neo-epitope corresponds to the cleavage site located between amino acids Val-Asp at positions 1709-1710 of human collagen type VII. The protease responsible for this cleavage is as yet unknown. The sequence was analyzed using BLAST and was found to be unique for the collagen type VII alpha-1 chain.

### Antibody

A monoclonal antibody was raised against the C-terminal neo-epitope amino acid sequence GPPGPPGRLV-COOH (SEQ ID NO: 1).

Briefly, four to six-week-old Balb/C mice were immunized subcutaneously with 200 μL emulsified antigen and 50 μg of a C7 synthetic peptide (KLH-CGG-GPPGPPGRLV, SEQ ID NO: 9) using Freund's incomplete adjuvant. Immunizations were performed every 2^{nd} week until stable sera titer levels were reached. The mouse with highest serum titer was selected for fusion. The mouse was rested for one month and then boosted intravenously with 50 μg C7 peptide in 100 μL 0.9% sodium chloride solution three days before isolation of the spleen for cell fusion. Mouse spleen cells were fused with SP2/0 myeloma fusion partner cells. The resulting hybridoma cells were cloned using a semi-solid medium method, transferred into 96-well microtiter plates for further growth and incubated in a CO₂ incubator. Standard limited dilution was used to promote monoclonal growth.

The sequence and CDRs of the selected monoclonal antibody were determined. The sequence of the chains are as follows (CDRs underlined and in bold; N-terminus signal peptide and C-terminus Constant region in italics):

Heavy Chain Sequence
(SEQ ID NO: 24)
MGWSWIFLFLLSGTAGVLSEVQLQQSGPELVKPGASVKISCKTSGYTFIE
NTMNWVKQSHGKSLEWIGGINPKIGGSIYNQKFQDKATLTVDKSSSTAYM
ELRSLTSDDSAVYYCARGIYDEGWHFDVWGAGTTVTVSSAKTTPPSVYPL
APGSAAQTNSMVTLGCLVKGYFPEPVTVTWNSGSLSSGVHTFPAVLQSDL
YTLSSSVTVPSSTWPSETVTCNVAHPASSTKVDKKIVPRDCGCKPCICTV
PEVSSVFIFPPKPKDVLTITLTPKVTCVVVDISKDDPEVQFSWFVDDVEV
HTAQTQPREEQFNSTFRSVSELPIMHQDWLNGKEFKCRVNSAAFPAPIEK
TISKTKGRPKAPQVYTIPPPKEQMAKDKVSLTCMITDFFPEDITVEWQWN
GQPAENYKNTQPIMDTDGSYFVYSKLNVQKSNWEAGNTFTCSVLHEGLHN
HHTEKSLSHSPGK
Light Chain Sequence
(SEQ ID NO: 25)
MRFSAQLLGLLVLWIPGSTADIVMTQAAFSNPVTLGTSASISCRSSQSLL
YSNGITYLYWYLQKPGQSPQLLIYQMSNLASGVPDRFSSSGSGTDFTLRI
SRVEAEDVGVYYCAQNLELPWTFGGGTKLEIKRADAAPTVSIFPPSSEQL
TSGGASVVCFLNNFYPKDINVKWKIDGSERQNGVLNSWTDQDSKDSTYSM
SSTLTLTKDEYERHNSYTCEATHKTSTSPIVKSFNRNEC

### ELISA

A competitive ELISA using the monoclonal antibody raised against C7 was performed using the following procedure:

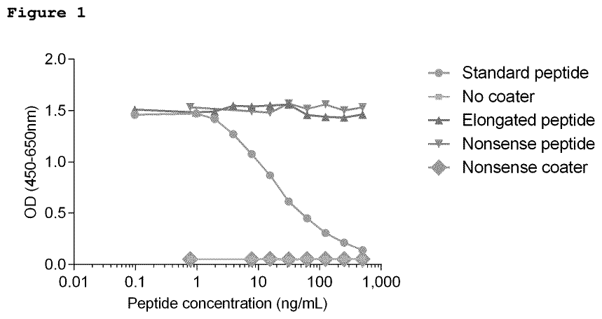

Streptavidin-coated plates were coated with 100 μL/well of 2.5 ng/mL biotin-labeled peptide (Biotin-KKGPPGPPGRLV, SEQ ID NO: 10) diluted in assay buffer (50 mM TBS-BTB, 2 g/L NaCl, pH 8.0) and incubated at 20° C., 300 rpm shaking for 30 minutes. Plates were washed five times in washing buffer (20 nM TRIS, 50 mM NaCl, pH 7.2). Sample or standard peptide (20 μL) was added in double determinations and followed immediately by addition of 100 μL/well of 200 ng/mL HRP-labeled monoclonal antibody diluted in assay buffer and plates were incubated at 20° C., 300 rpm shaking for 3 hours. The standard peptide was a synthetic peptide (GPPGPPGRLV, SEQ ID NO: 1) with a starting concentration of 125 ng/ml and diluted 2-fold to create an 11 points calibration curve (FIG. 1). After incubation, plates were washed five times in washing buffer. A volume of 100 μL 3,3′, 5,5′-tetramethylbenzidine (TMB) was added and incubated for 15 min at 20° C. in the dark. To stop the enzyme reaction of TMB, 100 mL 0.1% sulphuric acid was added and the absorbance was measured at 450 nm with 650 nm as the reference using an ELISA reader. A calibration curve was plotted using a 4-parametric mathematical fit model. Each ELISA plate included both kit control and in-house quality control samples to monitor inter-assay variation. All samples were measured within the range of the assay. All samples below the lower limit of detection (LLOD) were assigned the value of LLOD.

The technical characteristics of the C7 ELISA are as follows:

| Technical characteristics | Results |
|---|---|
| Biological matrix | Human serum |
| | (undiluted measurements) |
| Measurement range | 1.5-105.6 ng/mL |
| Normal range of healthy | 3.5 ng/mL |
| serum | |
| Inter-assay variation | 13% (accepted if <15%) |
| Intra-assay variation | 9% (accepted if <10%) |
| Dilution recovery | Accepted |
| | (undiluted to 1:8) |
| Spiking recovery | 166% (peptide in serum) |
| | 131% (serum in serum) |
| Analyte stability | Accepted |
| | (freeze/thaw and storage) |

The ELISA was shown to be specific for the cleavage site as reactivity was seen towards the standard peptide but not to an elongated peptide (GPPGPPGRLVD, SEQ ID NO: 11), indicating that the assay does not recognize intact collagen type VII protein (FIG. 1).

### Clinical Utility

### COPD

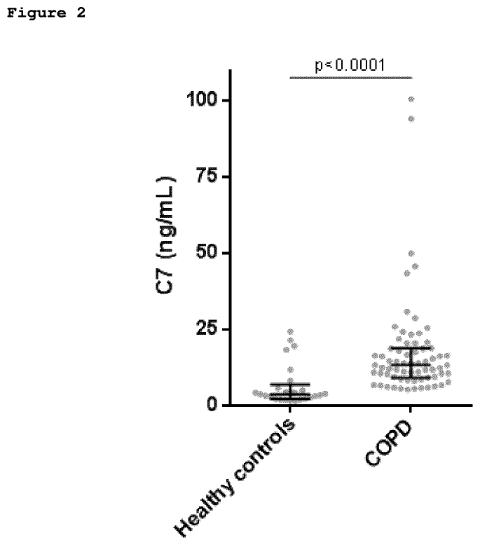

Serum levels of C7 were significantly elevated in a cohort of 68 patients with COPD when compared to healthy donors (FIG. 2).

These results show the utility of the C7 assay in identifying COPD, and may prove useful in evaluating COPD, for example as a diagnostic or prognostic tool.

### Systemic Sclerosis

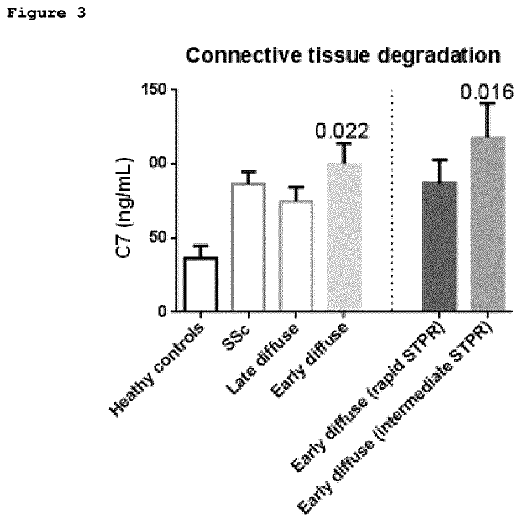

Serum levels of C7 were significantly elevated in 20 patients with early diffuse systemic sclerosis when compared to healthy control (p=0.022) (FIG. 3). The early stage of systemic sclerosis is associated with high disease activity, whereas the late stage patients are progressing slowly. In the group of patients with early diffuse disease, a subpopulation with intermediate progression rate (defined by the skin thickness progression rate) had significantly elevated levels when compared to controls (p=0.016).

The elevated level of C7 in early stage systemic sclerosis when compared to late stage systemic sclerosis suggests that the C7 assay may be capable of differentiating between early and late stages of systemic sclerosis, thereby providing a potentially useful diagnostic and/or prognostic tool for evaluating systemic sclerosis.

### Example 2 (“NB677” Assay)

The signal peptide in collagen type VII alpha-1 is found at amino acids 1-16 [12]. The N-terminal neo-epitope sequence that is formed by cleavage of the signal peptide (17 ‘. EAPRVRAQHR’26) was analyzed using BLAST and was found to be unique for the collagen type VII alpha-1 chain.

Following the success of the “C7” assay for COPD, it is postulated that this unique collagen type VII alpha-1 neo-epitope may also be useful in the identification and/or evaluation of COPD and/or systemic sclerosis.

### Antibody

Accordingly, a monoclonal antibody was raised against the N-terminal neo-epitope amino acid sequence H₂N-EAPRVRAQHR (SEQ ID NO: 4).

Briefly, four to six-week-old Balb/C mice were immunized subcutaneously with 200 μL emulsified antigen and 50 μg of a NB677 synthetic peptide (EAPRVRAQHR-GGC-KLH, SEQ ID NO: 12) using Freund's incomplete adjuvant. Immunizations were performed every 2^{nd} week until stable sera titer levels were reached. The mouse with highest serum titer was selected for fusion. The mouse was rested for one month and then boosted intravenously with 50 μg NB677 peptide in 100 μL 0.9% sodium chloride solution three days before isolation of the spleen for cell fusion. Mouse spleen cells were fused with SP2/0 myeloma fusion partner cells. The resulting hybridoma cells were cloned using a semi-solid medium method, transferred into 96-well microtiter plates for further growth and incubated in a CO₂ incubator. Standard limited dilution was used to promote monoclonal growth.

### ELISA

A competitive ELISA using the monoclonal antibody was performed using the following procedure:

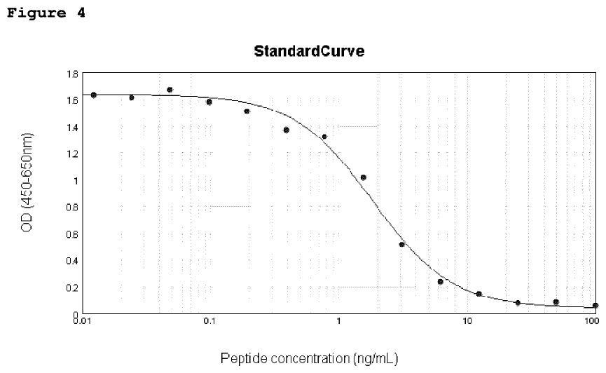

Streptavidin-coated plates were coated with 100 μL/well of 2.0 ng/mL biotin-labeled peptide (EAPRVRAQHR-Lys-Biotin, SEQ ID NO: 13) diluted in coating buffer (50 mM PBS-BTE, 8 g/L NaCl, 10% sorbitol) and incubated at 20° C., 300 rpm shaking for 30 minutes. Plates were washed five times in washing buffer (20 nM TRIS, 50 mM NaCl, pH 7.2). Standard peptide (20 μL) was added in double determinations and followed immediately by addition of 100 μL/well of 120 ng/ml monoclonal antibody diluted in assay buffer (25 mM PBS-BTB, 8 g/L NaCl) and plates were incubated at 4° C., 300 rpm shaking for 20 hours. The standard peptide was a synthetic peptide (EAPRVRAQHR, SEQ ID NO: 4) with a starting concentration of 100 ng/ml and diluted 2-fold to create a calibration curve (FIG. 4). After incubation, plates were washed five times in washing buffer. 100 μL/well of secondary HRP-labeled antibody (rabbit anti-mouse IgG) was added diluted 1:3000 in assay buffer and plates were incubated at 20° C., 300 rpm shaking for 1 hour. A volume of 100 μL 3,3′, 5,5′-tetramethylbenzidine (TMB) was added and incubated for 15 min at 20° C. in the dark. To stop the enzyme reaction of TMB, 100 mL 0.1% sulphuric acid was added and the absorbance was measured at 450 nm with 650 nm as the reference using an ELISA reader. A calibration curve was plotted using a 4-parametric mathematical fit model. The monoclonal antibody directed to the collagen type VII alpha 1 N-terminal neo-epitope has been confirmed to recognize the desired sequence, assessed by the reactivity to the standard peptide.

### Example 3

The C7 ELISA was evaluated in a second, larger cohort of patients with systemic sclerosis (SSc). The C7 ELISA was re-calibrated (compared to the previous example) to improve accuracy of the assessments in human serum.

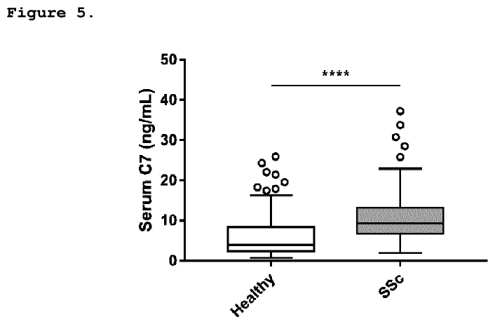

Results: The biological relevance of the C7 ELISA was evaluated by comparing serum levels in healthy donors (n=70) with patients with SSc (n=119). Data are shown in FIG. 5. Median serum C7 level was significantly elevated in patients with SSc (9.3 ng/mL [IQR 6.7-13.2]) as compared with healthy donors (3.9 ng/mL [IQR 2.3-8.3 ng/mL]; p<0.0001).

The clinical data support that serum C7 levels are elevated in patients with SSc

In conclusion, the novel assays described herein utilise antibodies specific for an N- or C-terminal neo-epitope of collagen VII alpha 1. To the best of our knowledge, this is the first time that collagen type VII has been associated with COPD. Accordingly, it is envisaged that these assays may be used for assessing COPD as well as systemic sclerosis.

In this specification, unless expressly otherwise indicated, the word ‘or’ is used in the sense of an operator that returns a true value when either or both of the stated conditions is met, as opposed to the operator ‘exclusive or’ which requires that only one of the conditions is met. The word ‘comprising’ is used in the sense of ‘including’ rather than in to mean ‘consisting of’. All prior teachings acknowledged above are hereby incorporated by reference. No acknowledgement of any prior published document herein should be taken to be an admission or representation that the teaching thereof was common general knowledge in Australia or elsewhere at the date hereof.

### REFERENCES

[1] Chung, H. J. and J. Uitto. 2010. Type VII collagen: the anchoring fibril protein at fault in dystrophic epidermolysis bullosa. Dermatol.Clin. 28:93-105.
[2] Chen, M., G. H. Kim, L. Prakash, and D. T. Woodley. 2012. Epidermolysis bullosa acquisita: autoimmunity to anchoring fibril collagen. Autoimmunity 45:91-101.
[3] Hundorfean, G., M. F. Neurath, and C. Sitaru. 2010. Autoimmunity against type VII collagen in inflammatory bowel disease. J.Cell Mol.Med. 14:2393-2403.
[4] Gammon, W. R., D. T. Woodley, K. C. Dole, and R. A. Briggaman. 1985. Evidence that anti-basement membrane zone antibodies in bullous eruption of systemic lupus erythematosus recognize epidermolysis bullosa acquisita autoantigen. J.Invest Dermatol. 84:472-476.
[5] Rudnicka, L., J. Varga, A. M. Christiano, R. V. Iozzo, S. A. Jimenez, and J. Uitto. 1994. Elevated expression of type VII collagen in the skin of patients with systemic sclerosis. Regulation by transforming growth factor-beta. J.Clin. Invest 93:1709-1715.
[6] Evans, M. J., M. V. Fanucchi, L. A. Miller, M. A. Carlson, S. J. Nishio, and D. M. Hyde. 2010. Reduction of collagen VII anchoring fibrils in the airway basement membrane zone of infant rhesus monkeys exposed to house dust mite. Am.J.Physiol Lung Cell Mol.Physiol 298:L543-L547.
[7] Chen, M., L. S. Chan, X. Cai, E. A. O'Toole, J. C. Sample, and D. T. Woodley. 1997. Development of an ELISA for rapid detection of anti-type VII collagen autoantibodies in epidermolysis bullosa acquisita. J.Invest Dermatol. 108:68-72.
[8] Saleh, M. A., K. Ishii, Y. J. Kim, A. Murakami, N. Ishii, T. Hashimoto, E. Schmidt, D. Zillikens, Y. Shirakata, K. Hashimoto, et al. 2011. Development of NC1 and NC2 domains of type VII collagen ELISA for the diagnosis and analysis of the time course of epidermolysis bullosa acquisita patients. J.Dermatol.Sci 62:169-175.
[9] Kim, J. H., Y. H. Kim, S. Kim, E. B. Noh, S. E. Kim, A. Vorobyev, E. Schmidt, D. Zillikens, and S. C. Kim. 2013. Serum levels of anti-type VII collagen antibodies detected by enzyme-linked immunosorbent assay in patients with epidermolysis bullosa acquisita are correlated with the severity of skin lesions. J.Eur. Acad Dermatol. Venereol. 27:e224-e230.
[10] Recke, A., C. Sitaru, G. Vidarsson, M. Evensen, M. T. Chiriac, R. J. Ludwig, and D. Zillikens. 2010. Pathogenicity of IgG subclass autoantibodies to type VII collagen: induction of dermal-epidermal separation. J.Autoimmun. 34:435-444.
[11] Sakai, L. Y., D. R. Keene, N. P. Morris, and R. E. Burgeson. 1986. Type VII collagen is a major structural component of anchoring fibrils. J.Cell Biol. 103:1577-1586.
[12] Uniprot. http://www. uniprot.org/uniprot/Q02388. Accessed 2015.

1. An antibody reactive with a fragment of collagen type VII alpha 1 comprising an N- or C-terminal neo-epitope, wherein said antibody binds to the N- or C-terminal neo-epitope.

2. An antibody as claimed in claim 1, wherein the antibody is a monoclonal antibody.

3. An antibody as claimed in claim 1 or 2, wherein said N- or C-terminal neo-epitope is comprised in a non-collagenous amino-terminal domain of collagen type VII alpha 1 or is comprised in a central collagenous triple helical domain of collagen type VII alpha 1.

4. An antibody as claimed in claims 1 to 3, wherein said antibody binds to a C-terminal neo-epitope comprised in the amino acid sequence GPPGPPGRLV-COOH (SEQ ID NO: 1).

5. An antibody as claimed in claims 1 to 3, wherein said antibody binds to a C-terminal neo-epitope comprising the amino acid sequence PPGRLV-COOH (SEQ ID NO: 2).

6. An antibody as claimed in claim 4 or 5, wherein said antibody does not recognise or bind elongated amino acid sequence GPPGPPGRLVX-COOH (SEQ ID NO: 3), wherein X is one or more amino acids of the sequence of collagen type VII alpha 1.

7. An antibody as claimed in claims 1 to 3, wherein said antibody binds to an N-terminal neo-epitope comprised in the amino acid sequence H₂N-EAPRVRAQHR (SEQ ID NO: 4).

8. An antibody as claimed in claims 1 to 3, wherein said antibody binds to an N-terminal neo-epitope comprising the amino acid sequence H₂N-EAPRVR (SEQ ID NO: 5).

9. An antibody as claimed in claim 7 or 8, wherein said antibody does not recognise or bind elongated amino acid sequence H₂N-XEAPRVRAQHR (SEQ ID NO: 6), wherein X is one or more amino acids of the sequence of collagen type VII alpha 1.

10. A method of immunoassay for detecting in a biological sample a fragment of collagen type VII alpha 1 comprising an N- or C-terminal neo-epitope, said method comprising contacting said biological sample comprising said fragment of collagen type VII alpha 1 comprising said N- or C-terminal neo-epitope with an antibody as claimed in any preceding claim, and determining the amount of binding of said antibody.

11. A method as claimed in claim 10, wherein said method is used to quantify the amount of the fragment of collagen type VII alpha 1 comprising said N- or C-terminal neo-epitope in biofluids.

12. A method as claimed in claim 11, wherein said biofluid is serum, plasma, bronchoalveolar lavage fluid, sputum, exhaled breath or urine.

13. A method as claimed in claim 10, wherein said immunoassay is a competition assay or a sandwich assay.

14. A method as claimed in claim 10, wherein said immunoassay is a radioimmunoassay or an enzyme-linked immunosorbent assay.

15. A method as claimed in claim 11, further comprising correlating the quantity of the fragment of collagen type VII alpha 1 comprising an N- or C-terminal neo-epitope determined by said method with standard collagen type VII related disease samples of known disease severity to evaluate the severity of a collagen type VII related disease.

16. A method as claimed in claim 15, wherein the collagen type VII related disease is chronic obstructive pulmonary disease (COPD) or systemic sclerosis.

17. A peptide, wherein the peptide has an N-terminal amino acid sequence corresponding to an amino acid sequence of an N-terminal neo-epitope of a fragment of collagen type VII alpha 1 comprising said N-terminal neo-epitope, or wherein the peptide has a C-terminal amino acid sequence corresponding to an amino acid sequence of an C-terminal neo-epitope of a fragment of collagen type VII alpha 1 comprising said C-terminal neo-epitope.

18. A peptide as claimed in claim 17, wherein the N-terminal neo-epitope amino acid sequence is EAPRVRAQHR (SEQ ID NO: 4) or EAPRVR (SEQ ID NO: 5).

19. A peptide as claimed in claim 17, wherein the C-terminal neo-epitope amino acid sequence is GPPGPPGRLV (SEQ ID NO: 1) or PPGRLV (SEQ ID NO: 2).

20. A peptide as claimed in claims 17 to 19, wherein the peptide is conjugated to biotin.

21. An assay kit for determining the quantity of a fragment of collagen type VII alpha 1 comprising an N- or C-terminal neo-epitope in a biological sample, the kit comprising an antibody as claimed in any of claims 1 to 9 and at least one of:
a streptavidin coated 96 well plate
a biotinylated peptide of claims 17-19, with an optional linker located between the biotin residue and the peptide
a biotinylated secondary antibody for use in a sandwich immunoassay
a calibrator peptide of claims 17-19
an antibody HRP labeling kit
an antibody radiolabeling kit
an assay visualization kit

22. An assay kit as claimed in claim 20, comprising a biotinylated peptide Biotin-L-GPPGPPGRLV (SEQ ID NO: 7), wherein L is an optional linker, and a calibrator peptide comprising the C-terminal sequence GPPGPPGRLV-COOH (SEQ ID NO: 1).

23. An assay kit as claimed in claim 20, comprising a biotinylated peptide EAPRVRAQHR-L-Biotin (SEQ ID NO: 8), wherein L is an optional linker, and a calibrator peptide comprising the N-terminal sequence H₂N-EAPRVRAQHR (SEQ ID NO: 4).
